(19) United States
(12) Patent Application Publication
(10) Pub. No.: US 2019/0254569 A1
(43) Pub. Date: Aug. 22, 2019

(54) MOVEMENT ABILITY EVALUATING APPARATUS, MOVEMENT ABILITY EVALUATING SYSTEM, MOVEMENT ABILITY EVALUATING PROGRAM, AND MOVEMENT ABILITY EVALUATING METHOD

(71) Applicant: SUMITOMO ELECTRIC INDUSTRIES, LTD., Osaka (JP)

(72) Inventors: Yusuke ASADA, Osaka (JP); Hideaki TOSHIOKA, Osaka (JP)

(73) Assignee: SUMITOMO ELECTRIC INDUSTRIES, LTD., Osaka (JP)

(21) Appl. No.: 16/333,797

(22) PCT Filed: Jun. 2, 2017
(86) PCT No.: PCT/JP2017/020634

§ 371 Date: Mar. 15, 2019

(30) Foreign Application Priority Data
Sep. 16, 2016 (JP) 2016-181572

(51) Int. Cl. A61B 5/11; A61B 5/00

(52) U.S. Cl. CPC A61B 5/112; A61B 5/4023; A61B 5/486; A61B 2562/0219; A61B 5/7214; A61B 5/0002; A61B 5/742

(57) ABSTRACT

A movement ability evaluating apparatus includes a communication unit and a control unit. The communication unit is configured to acquire front-back acceleration, right-left acceleration, and up-down acceleration during movement of a subject measured by an acceleration sensor attached to the waist of the subject. The control unit is configured to evaluate the movement ability of the subject, based on temporal change of the front-back acceleration, the right-left acceleration, and the up-down acceleration acquired by the communication unit. The movement ability of the subject includes at least one of front-back balance, right-left balance, and weight shift during movement of the subject.

### TECHNICAL FIELD

The present disclosure relates to a movement ability evaluating apparatus, a movement ability evaluating system, a movement ability evaluating program, and a movement ability evaluating method. The subject application claims the priority based on Japanese Patent Application No. 2016-181572 filed on Sep. 16, 2016 with the Japan Patent Office, the entire contents of which are hereby incorporated by reference.

### BACKGROUND ART

Evaluation of the movement ability of a subject has been conducted as appropriate. The evaluation of the movement ability can be used, for example, for predicting falls.

For example, Japanese Patent Laying-Open No. 2008-229266 (PTL 1) discloses a technique that measures temporal change in waist acceleration including up-down acceleration that is acceleration in the up-down direction of the subject's waist during walking, front-back acceleration that is acceleration in the front-back direction of the waist, and right-left acceleration that is acceleration in the right-left direction of the waist, and detects the subject's ability to walk based on the measured values.

Japanese Patent Laying-Open No. 2009-89740 (PTL 2) discloses a technique that identifies actions of a subject, such as walking, running, ascending stairs, and descending stair, based on the magnitude and the direction of acceleration of the body axis in the subject's front-back direction, right-left direction, and up-down direction that are detected at certain time intervals.

Japanese Patent Laying-Open No. 2010-172481 (PTL 3) discloses a technique that sets a risk indicator for use in evaluating the risk of falling of a subject by calculating a statistic related to acceleration, based on accelerations produced in the up-down direction, the right-left direction, and the front-back direction during walking or during exercise at a predetermined body part (right and left toes, right and left knee joints, and waist) of a subject and measured by an acceleration sensor attached to the body part, and analyzing the receiver operating characteristic of the calculated statistic.

### CITATION LIST

### Patent Literature

PTL 1: Japanese Patent Laying-Open No. 2008-229266
PTL 2: Japanese Patent Laying-Open No. 2009-89740
PTL 3: Japanese Patent Laying-Open No. 2010-172481

### SUMMARY OF INVENTION

A movement ability evaluating apparatus according to an aspect of the present invention is configured to evaluate movement ability of a subject and includes a communication unit and a control unit. The communication unit is configured to acquire front-back acceleration, right-left acceleration, and up-down acceleration during movement of the subject measured by an acceleration sensor attached to waist of the subject. The control unit is configured to evaluate the movement ability of the subject, based on temporal change of the front-back acceleration, the right-left acceleration, and the up-down acceleration acquired by the communication unit. The movement ability of the subject includes at least one of front-back balance, right-left balance, and weight shift during movement of the subject.

A movement ability evaluating system according to an aspect of the present invention includes an acceleration sensor attached to waist of a subject, and a movement ability evaluating apparatus configured to evaluate movement ability of the subject, based on a signal output by the acceleration sensor. The movement ability evaluating apparatus includes a communication unit and a control unit. The communication unit is configured to acquire front-back acceleration, right-left acceleration, and up-down acceleration during movement of the subject measured by the acceleration sensor. The control unit is configured to evaluate the movement ability, based on temporal change of the front-back acceleration, the right-left acceleration, and the up-down acceleration acquired by the communication unit. The movement ability includes at least one of front-back balance, weight shift, and right-left balance during movement of the subject.

A movement ability evaluating program according to an aspect of the present invention is a program for causing a computer to execute a process of evaluating movement ability of a subject. The movement ability includes at least one of front-back balance, weight shift, and right-left balance during movement of the subject. The movement ability evaluating program causes the computer to execute the steps of: acquiring front-back acceleration, right-left acceleration, and up-down acceleration during movement of the subject measured by an acceleration sensor attached to waist of the subject; and evaluating the movement ability, based on temporal change of the acquired front-back acceleration, right-left acceleration, and up-down acceleration.

A movement ability evaluating method according to an aspect of the present invention evaluates movement ability of a subject. The movement ability evaluating method includes acquiring front-back acceleration, right-left acceleration, and up-down acceleration during movement of the subject measured by an acceleration sensor attached to waist of the subject, and evaluating the movement ability, based on temporal change of the acquired front-back acceleration, right-left acceleration, and up-down acceleration. The movement ability includes at least one of front-back balance, weight shift, and right-left balance during movement of the subject.

### BRIEF DESCRIPTION OF DRAWINGS

FIG. 1 is a diagram schematically showing a configuration of a movement ability evaluating system according to a first embodiment.

FIG. 2 is a diagram schematically showing a hardware configuration of the movement ability evaluating system according to the first embodiment.

FIG. 3 is a diagram schematically showing a functional configuration of an acceleration sensor according to the first embodiment.

FIG. 4 is a diagram schematically showing a functional configuration of a movement ability evaluating apparatus according to the first embodiment.

FIG. 5 is a diagram showing the relation between a human walking cycle and front-back acceleration, up-down acceleration, and right-left acceleration during walking.

FIG. 6 is a flowchart for explaining movement ability evaluation executed by the movement ability evaluating system according to the first embodiment.

FIG. 7 is a flowchart for explaining the procedure of evaluating movement ability at step S18 in FIG. 6.

FIG. 8 is a diagram for explaining the process at steps S32 and S33 in FIG. 7.

FIG. 9 is a diagram for explaining the process at step S34 in FIG. 7.

FIG. 10 is a diagram for explaining the process at step S35 in FIG. 7.

FIG. 11 is a diagram for explaining the process at step S36 in FIG. 7.

FIG. 12 is a diagram for explaining the process at step S36 in FIG. 7.

FIG. 13 is a diagram showing a display example of the evaluation result of movement ability.

FIG. 14 is a diagram showing a display example of exercise advice.

FIG. 15 is a flowchart for explaining the procedure of evaluating movement ability at step S18 in FIG. 6.

FIG. 16 is a diagram for explaining the process at step S43 in FIG. 15.

FIG. 17 is a diagram for explaining the process at step S43 in FIG. 15.

FIG. 18 is a diagram for explaining the process at step S43 in FIG. 15.

FIG. 19 is a diagram schematically showing another configuration of the movement ability evaluating system.

### DETAILED DESCRIPTION

### Problem to be Solved by the Present Disclosure

With the technique disclosed in PTL 1, walking speed, stride length, walking pace, etc. during walking in different walking modes are detected as the ability to walk of the subject, and the risk of falling of the subject is determined from these detected values.

Typically, the falls of, for example, elderly people are closely related to reduction in motor functions, such as muscle strength reduction, balance ability reduction, limited range of joint motion, bendability reduction, and posture change. Reduction in such motor functions makes it difficult to keep balance during walking or impairs proper weight shift, leading to the likelihood of falling during movement.

Unfortunately, although the technique in PTL 1 estimates the subject's ability to walk from walking speed, stride length, walking pace, etc., it is difficult to properly evaluate, for example, balance ability and weight shift ability. Consequently, it is impossible to precisely determine the risk of falling of the subject.

The technique disclosed in PTL 2 measures the acceleration of the body axis of a subject. However, this technique is aimed to accurately calculate calories corresponding to an action by precisely identifying a human action using the measured value, and there is no mention about evaluation of reduction of physical functions as described above.

The technique disclosed in PTL 3 calculates a statistic related to acceleration by averaging the accelerations produced for a certain time in which a walking motion or an exercise motion is performed. It is therefore difficult to properly evaluate, for example, balance ability and weight shift ability of a subject for a certain time from the calculated statistic.

An object of an aspect of the present invention is to provide a movement ability evaluating apparatus, a movement ability evaluating system, a movement ability evaluating program, and a movement ability evaluating method capable of properly evaluating the movement ability of a subject.

### Advantageous Effect of the Present Disclosure

According to the foregoing, the movement ability of a subject can be properly evaluated.

### Description of Embodiments of the Present Invention

First of all, embodiments of the present invention are listed below.

(1) A movement ability evaluating apparatus according to an aspect of the present invention evaluates movement ability of a subject. The movement ability evaluating apparatus includes a communication unit and a control unit. The communication unit is configured to acquire front-back acceleration, right-left acceleration, and up-down acceleration during movement of the subject measured by an acceleration sensor attached to waist of the subject. The control unit is configured to evaluate the movement ability, based on temporal change of the front-back acceleration, the right-left acceleration, and the up-down acceleration acquired by the communication unit. The movement ability includes at least one of front-back balance, right-left balance, and weight shift during movement of the subject.

According to the foregoing, the movement ability of a subject can be properly evaluated by using at least one of front-back balance, right-left balance, and weight shift of the subject during movement as an indicator for evaluating the movement ability of the subject. With this configuration, the risk of falling of the subject can be determined precisely.

(2) Preferably, in the movement ability evaluating apparatus described in (1) above, the control unit calculates an indicator indicating the front-back balance, based on a temporal waveform of the front-back acceleration.

According to the foregoing, change in front-back direction of the body center of gravity during movement of the subject can be quantitatively evaluated from the temporal waveform of front-back acceleration. The front-back balance during movement of the subject thus can be evaluated.

Preferably, the control unit calculates an indicator indicating the front-back balance, based on a distribution state of forward acceleration and backward acceleration in a temporal waveform of the front-back acceleration in at least one walking cycle.

In this manner, the front-back balance during movement of the subject can be quantitatively evaluated.

(3) Preferably, in the movement ability evaluating apparatus described in (1) above, the control unit searches for a heel contact time and a mid stance time of one foot of the subject in a temporal waveform of the front-back acceleration in one walking cycle. The control unit calculates an indicator indicating the weight shift in the one foot, based on a temporal waveform of the up-down acceleration in a period of time from the heel contact time to the mid stance time.

According to the foregoing, change in up-down direction of the body center of gravity of the subject after the heel touches the ground can be quantitatively evaluated from a temporal waveform of the up-down acceleration in a period of time from a heel contact time to a mid stance time. With this configuration, the weight shift in the sole of the supporting leg can be evaluated.

Preferably, the control unit searches for a stepping motion time immediately after heel contact and a stepping motion time immediately after ball of foot contact in a temporal waveform of the up-down acceleration in a period of time from a heel contact time to a mid stance time. The control unit calculates an indicator indicating the weight shift, based on a temporal waveform of the up-down acceleration in the vicinity of a stepping motion time immediately after heel contact and a stepping motion time immediately after ball of foot contact. In this manner, change in up-down direction of the body center of gravity of the subject due to a step motion immediately after heel contact and immediately after ball of foot contact can be quantitatively evaluated.

More preferably, the control unit calculates an indicator indicating the weight shift, based on the ratio between a value of integral obtained by time-integrating upward acceleration in a period of time from a heel contact time to a stepping motion time immediately after ball of foot contact and a value of integral obtained by time-integrating upward acceleration in a period of time from a stepping motion time immediately after ball of foot contact to a mid stance time. In this manner, the weight shift in the sole of the supporting leg can be quantitatively evaluated.

(4) Preferably, in the movement ability evaluating apparatus described in (1) above, the control unit searches for a right heel contact time, a right mid stance time, a left heel contact time, and a left mid stance time of the subject in a temporal waveform of the front-back acceleration in one walking cycle. The control unit calculates an indicator indicating the right-left balance, based on a temporal waveform of leftward acceleration in a period of time from the right heel contact time to the right mid stance time and a temporal waveform of rightward acceleration from the left heel contact time to the left mid stance time.

According to the foregoing, change in right-left direction of the body center of gravity of the subject after the heel touches the ground can be quantitatively evaluated from a temporal waveform of right-left acceleration in a period of time from a heel contact time to a mid stance time. With this configuration, the right-left balance during movement of the subject can be evaluated.

Preferably, the control unit calculates an indicator indicating the right-left balance, based on the ratio between a value of integral obtained by time-integrating leftward acceleration in a period of time from a right heel contact time to a right mid stance time and a value of integral obtained by time-integrating rightward acceleration in a period of time from a left heel contact time to a left mid stance time. In this manner, change in left direction of the body center of gravity of the subject due to right heel contact can be quantitatively calculated from a temporal waveform of leftward acceleration in a period of time from a right heel contact time to a right mid stance time. In addition, change in right direction of the body center of gravity of the subject due to left heel contact can be quantitatively calculated from a temporal waveform of rightward acceleration in a period of time from a left heel contact time to a left mid stance time. Therefore, the right-left balance during movement of the subject can be quantitatively evaluated.

(5) Preferably, in the movement ability evaluating apparatus described in (1) above, the control unit calculates an indicator indicating the front-back balance, based on an autocorrelation function of the front-back acceleration.

According to the foregoing, front-back balance during movement of the subject can be evaluated by capturing the periodicity of temporal change of front-back acceleration during movement using the autocorrelation function of front-back acceleration. This configuration can reduce computation processes in the control unit, compared with the configuration in which front-back balance is evaluated by searching for the time when the subject is performing a certain motion from a temporal waveform of front-back acceleration. This achieves faster computation. In other words, while fast computation is achieved, an inexpensive computer can be used, thereby simplifying the system configuration.

Preferably, the control unit calculates an indicator indicating the front-back balance, based on a deviation of a valley portion positioned between an origin and a first peak position of an autocorrelation function of the front-back acceleration from an approximate curve obtained by approximating the valley portion to a quadric curve. With this configuration, the front-back balance during movement of the subject can be quantitatively evaluated from the magnitude of deviation.

(6) Preferably, in the movement ability evaluating apparatus described in (1) above, the control unit calculates an indicator indicating the weight shift, based on an autocorrelation function of the up-down acceleration.

According to the foregoing, the weight shift during movement of the subject can be evaluated by capturing the periodicity of temporal change of up-down acceleration during movement using the autocorrelation function of up-down acceleration. This configuration can reduce computation processes in the control unit, compared with the configuration in which the weight shift is evaluated by searching for the time when the subject is performing a certain motion from a temporal waveform of front-back acceleration.

Preferably, the control unit calculates an indicator indicating the weight shift, based on the ratio between a value at the origin and a value at a first peak position of an autocorrelation function of the up-down acceleration. In this manner, change in position of the body center of gravity due to a stepping motion immediately after heel contact and immediately after ball of foot contact can be captured from the autocorrelation function of the up-down acceleration, so that the weight shift during movement of the subject can be evaluated.

(7) Preferably, in the movement ability evaluating apparatus described in (1) above, the control unit calculates an indicator indicating the right-left balance, based on an autocorrelation function of the front-back acceleration and an autocorrelation function of the right-left acceleration.

According to the foregoing, the periodicity of temporal change of right-left acceleration during movement can be captured using the autocorrelation function of front-back acceleration and the autocorrelation function of right-left acceleration, so that the right-left balance during movement of the subject can be evaluated. This configuration can reduce computation processes in the control unit, compared with the configuration in which the time when the subject is performing a certain motion is searched for in the temporal waveform of front-back acceleration, and the right-left balance is evaluated based on the temporal waveform of right-left balance in a period of time specified by the found time of motion.

Preferably, the control unit searches for a first peak position and a second peak position of the autocorrelation function of the front-back acceleration. The control unit searches for a first value at a peak position corresponding to the first peak position and a second value at a peak position corresponding to the second peak position in the autocorrelation function of the right-left acceleration. The control unit calculates an indicator indicating the right-left acceleration, based on a ratio between the first value and the second value. With this configuration, the weight shift during movement of the subject can be evaluated by comparing two values of the autocorrelation function of right-left acceleration corresponding to two peak positions appearing in the autocorrelation function of front-back acceleration.

(8) Preferably, in the movement ability evaluating apparatus described in (1) to (7) above, the control unit determines exercise advice suitable for the subject, based on an indicator indicating the movement ability.

According to the foregoing, since the movement ability of a subject can be properly evaluated, exercise advice effective for improving the movement ability of a subject can be provided. The subject undergoes rehabilitation in accordance with the exercise advice to reduce the risk of falling of the subject in the future.

(9) Preferably, the movement ability evaluating apparatus described in (8) above further includes a display configured to display at least one of the evaluation result by the control unit and the exercise advice.

According to the foregoing, the user or the subject can easily check the movement ability of the subject and the exercise advice.

(10) A movement ability evaluating system according to an aspect of the present invention includes an acceleration sensor attached to waist of a subject and a movement ability evaluating apparatus configured to evaluate movement ability of the subject, based on a signal output by the acceleration sensor. The movement ability evaluating apparatus includes a communication unit and a control unit. The communication unit is configured to acquire front-back acceleration, right-left acceleration, and up-down acceleration during movement of the subject measured by the acceleration sensor. The control unit is configured to evaluate the movement ability, based on temporal change of the front-back acceleration, the right-left acceleration, and the up-down acceleration acquired by the communication unit. The movement ability includes at least one of front-back balance, weight shift, and right-left balance during movement of the subject.

According to the foregoing, the movement ability of the subject can be properly evaluated by using at least one of front-back balance, right-left balance, and weight shift of the subject during movement as an indicator for evaluating the movement ability of the subject. With this configuration, the risk of falling of the subject can be determined precisely.

(11) Preferably, in the movement ability evaluating system described in (10) above, the acceleration sensor includes a sensor unit and a signal processing circuit. The sensor unit is configured to measure front-back acceleration, right-left acceleration, and up-down acceleration produced at the waist of the subject. The signal processing circuit corrects a measured value of the sensor unit when the subject is standing still to a zero point of the front-back acceleration, the right-left acceleration, and the front-back acceleration. The signal processing circuit is further configured to acquire a measured value of the sensor unit at intervals of 1 ms to 200 ms during movement of the subject.

According to the foregoing, the front-back acceleration, the right-left acceleration, and the up-down acceleration produced during movement of the subject can be precisely measured by performing a zero-point correction for the sensor unit when the subject is standing still. With this configuration, the movement ability of the subject can be properly evaluated based on the measured value of the sensor unit.

(12) Preferably, in the movement ability evaluating system described in (10) above, the movement ability evaluating apparatus further includes a storage device configured to store the front-back acceleration, the right-left acceleration, and the up-down acceleration acquired by the communication unit and the evaluation result in the control unit. The acceleration sensor includes a transmitter and a memory. The transmitter is configured to transmit the measured value of the sensor unit acquired by the signal processing circuit to the communication unit. The memory is configured to save the measured value of the sensor unit acquired by the signal processing circuit. The signal processing circuit is configured to select one of the storage device and the memory in accordance with a signal from the movement ability evaluating apparatus to save the measured value of the sensor unit.

According to the foregoing, the movement ability can be evaluated in real time using the measured value by transmitting the measured value by the sensor unit to the movement ability evaluating apparatus and saving the measured value into the internal storage device in the movement ability evaluating apparatus. Alternatively, the measured value by the sensor unit may be stored in the internal memory of the acceleration sensor so that the movement ability can be evaluated later using the measured value stored in the memory. Alternatively, acceleration is measured over a few hours (or a few days) and the measured value is stored in the memory so that the movement ability of the subject as well as the exercise habit of the subject can be evaluated using the measured value.

(13) A movement ability evaluating program according to an aspect of the present invention is a program for causing a computer to execute a process of evaluating movement ability of a subject. The movement ability includes at least one of front-back balance, weight shift, and right-left balance during movement of the subject. The movement ability evaluating program causes the computer to execute the steps of: acquiring front-back acceleration, right-left acceleration, and up-down acceleration during movement of the subject measured by an acceleration sensor attached to waist of the subject, and evaluating the movement ability, based on temporal change of the acquired front-back acceleration, right-left acceleration, and up-down acceleration.

According to the foregoing, the movement ability of the subject can be properly evaluated by using at least one of front-back balance, right-left balance, and weight shift of the subject during movement as an indicator for evaluating the movement ability of the subject. With this configuration, the risk of falling of the subject can be determined precisely.

A computer-readable storage medium such as USB (Universal Serial Bus) memory, flexible disc, CD (Compact Disc), DVD. Blu-ray Disc (registered trademark). MO (Magneto-Optical disc), SD card, memory stick (registered trademark), magnetic disc, optical disc, magneto-optical disc, semiconductor memory, and magnetic tape can be used as a storage medium to store the movement ability evaluating program. A storage medium typically fixed in a system or a device, such as HDD (Hard Disc Drive) and SSD (Solid State Drive), may be used.

(14) A movement ability evaluating method according to an aspect of the present invention evaluates movement ability of a subject. The movement ability evaluating method includes: acquiring front-back acceleration, right-left acceleration, and up-down acceleration during movement of the subject measured by an acceleration sensor attached to waist of the subject; and evaluating the movement ability, based on temporal change of the acquired front-back acceleration, right-left acceleration, and up-down acceleration. The movement ability includes at least one of front-back balance, weight shift, and right-left balance during movement of the subject.

According to the foregoing, since the movement ability of the subject can be properly evaluated, the risk of falling of the subject can be determined precisely.

### Description of Embodiments

### First Embodiment

(Configuration of Movement Ability Evaluating System 100)

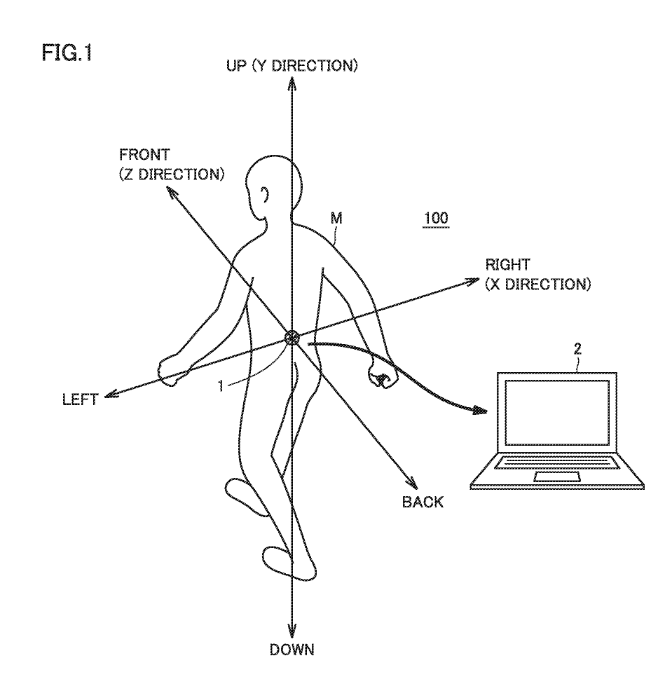

FIG. 1 is a diagram schematically showing a configuration of a movement ability evaluating system 100 according to a first embodiment. Movement ability evaluating system 100 according to the first embodiment is a system for evaluating the movement ability of a subject M. In the description of the subject application, the “movement ability” of subject M is the motor ability of subject M in movement (walking or running) and at least includes balance ability (front-back balance, right-left balance) and weight shift ability. In the description of the subject application, the “front-back balance” refers to balance in the front-back direction of the body center of gravity involved with movement. The “right-left balance” refers to balance in the right-left direction of the body center of gravity involved with movement. The “weight shift” refers to weight shift of the sole involved with movement.

As shown in FIG. 1, movement ability evaluating system 100 includes an acceleration sensor 1 and a movement ability evaluating apparatus 2. Acceleration sensor 1 and movement ability evaluating apparatus 2 communicate with each other by radio. Specifically, acceleration sensor 1 is connected to movement ability evaluating apparatus 2 in accordance with short-range wireless communication standards such as Bluetooth (registered trademark) and wireless LAN (Local Area Network) standards to transmit/receive data to/from movement ability evaluating apparatus 2.

Acceleration sensor 1 has a portable small casing and is attached to the waist of subject M. Preferably, acceleration sensor 1 is attached to the vicinity of third lumbar vertebra on the median line where the body center of gravity of subject M exists. For example, the casing of acceleration sensor 1 has a clip (not shown), and acceleration sensor 1 is attached by fastening the clip near the center of the lower back portion of the belt worn by subject M.

Acceleration sensor 1 is a three-axis acceleration sensor such as a MEMS (Micro Electro Mechanical System) sensor. Acceleration sensor 1 measures the accelerations in the right-left direction, the up-down direction, and the front-back direction during movement of subject M. In the following description, the acceleration in the right-left direction may be referred to as “right-left acceleration”, the acceleration in the up-down direction may be referred to as “up-down acceleration”, and the acceleration in the front-back direction may be referred to as “front-back acceleration”. The right-left direction for subject M is the X axis, the up-down direction is the Y axis, and the front-back direction is the Z axis.

Acceleration sensor 1 outputs the measured three-axis acceleration as measurement data to movement ability evaluating apparatus 2. Acceleration sensor 1 may be any device that can measure change of three-axis acceleration during movement of subject M. It is preferable that subject M moves barefoot in order to accurately measure change of three-axis acceleration during movement.

Movement ability evaluating apparatus 2 is an electronic device having a wireless communication function, and, for example, a personal computer, a tablet terminal, a smartphone, or the like can be used Movement ability evaluating apparatus 2 acquires front-back acceleration, right-left acceleration, and up-down acceleration during movement of subject M, from measurement data output by acceleration sensor 1. Movement ability evaluating apparatus 2 evaluates the movement ability of subject M, based on temporal change of the acquired front-back acceleration, right-left acceleration, and up-down acceleration.

(Hardware Configuration of Movement Ability Evaluating System)

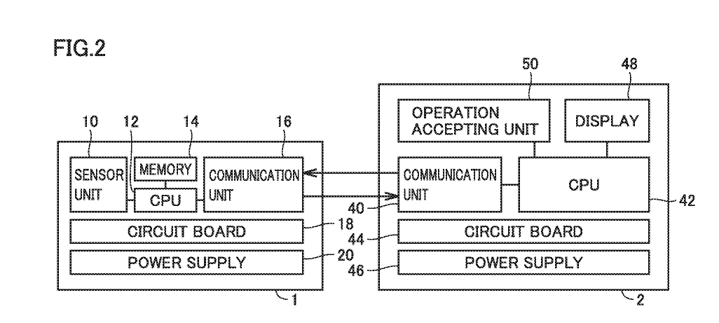

FIG. 2 is a diagram schematically showing a hardware configuration of movement ability evaluating system 100 according to the first embodiment.

As shown in FIG. 2, acceleration sensor 1 includes a sensor unit 10, a CPU (Central Processing Unit) 12, a memory 14, a communication unit 16, a circuit board 18, and a power supply 20.

Sensor unit 10 is a three-axis acceleration sensor and measures front-back acceleration, right-left acceleration, and up-down acceleration produced at the waist of subject M. Sensor unit 10 outputs an electrical signal indicating the measured acceleration to CPU 12.

CPU 12 controls the operation of acceleration sensor 1 by reading a program stored in advance and executing instructions included in the program. CPU 12 processes an electrical signal output from sensor unit 10 to generate measurement data from the acceleration measured by sensor unit 10.

Memory 14 is configured, for example, with a RAM (Random Access Memory) to store setting data for setting a variety of functions of acceleration sensor 1 and measurement data.

Communication unit 16 performs, for example, modulation/demodulation processing for transmitting/receiving a signal through a not-shown antenna so that acceleration sensor 1 communicates with movement ability evaluating apparatus 2 by radio. Specifically, communication unit 16 is a communication module including a tuner, a received signal strength calculation circuit, a cyclic redundancy check circuit, and a high frequency circuit. Communication unit 16 performs modulation/demodulation and frequency conversion of a radio signal transmitted/received by acceleration sensor 1 and applies a received signal to CPU 12.

Circuit board 18 is accommodated in the casing of acceleration sensor 1 and is populated with circuit components of each of sensor unit 10, CPU 12, memory 14, and communication unit 16.

Power supply 20 is a power storage device including a lithium ion battery. When a not-shown power switch is turned on, for example, by a user, power supply to a plurality of circuit components mounted on circuit board 18 is started.

Movement ability evaluating apparatus 2 includes a communication unit 40, a CPU 42, a circuit board 44, a power supply 46, a display 48, and an operation accepting unit 50.

Communication unit 40 performs, for example, modulation/demodulation processing for transmitting/receiving a signal through an antenna so that movement ability evaluating apparatus 2 communicates with other wireless devices including acceleration sensor 1. Communication unit 40 is a communication module including a tuner, a received signal strength calculation circuit, a cyclic redundancy check circuit, and a high frequency circuit. Communication unit 40 performs modulation/demodulation and frequency conversion of a radio signal transmitted/received by movement ability evaluating apparatus 2 and applies a received signal to CPU 42.

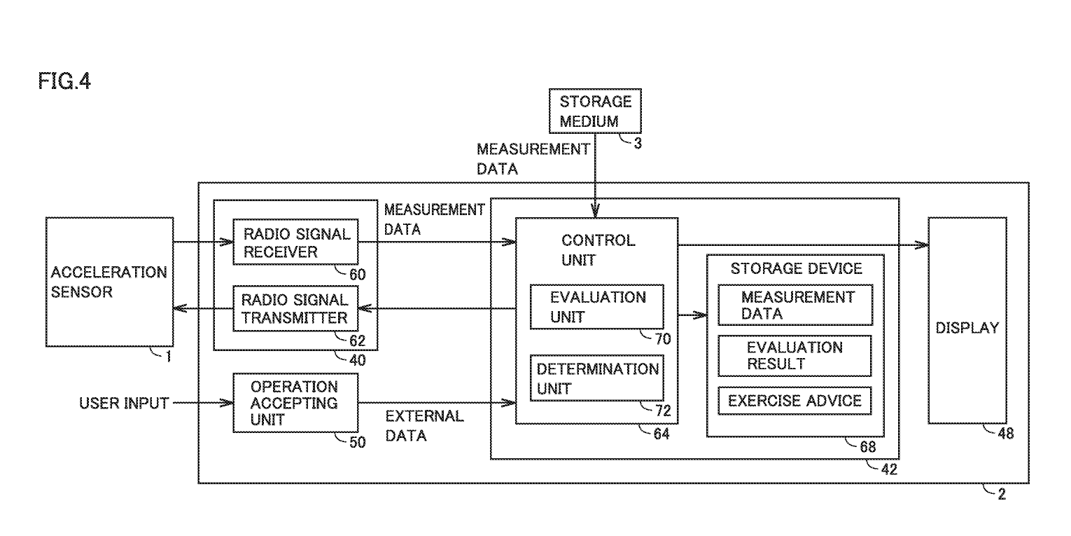

CPU 42 controls the operation of movement ability evaluating apparatus 2 by reading a program stored in storage device 68 (see FIG. 4) and executing an instruction included in the program. The program includes a movement ability evaluating program. CPU 42 executes the movement ability evaluating program to evaluate the movement ability of subject M based on measurement data transmitted from communication unit 40. CPU 42 determines exercise advice suitable for subject M based on the evaluation result of movement ability. The details of CPU 42 will be described later.

Operation accepting unit 50 accepts an input operation by the user. Operation accepting unit 50 outputs a signal indicating the operation content to CPU 42 in accordance with the user operation. Operation accepting unit 50 may be a touch panel provided on display 48 or may be other physical operation keys such as keyboard.

Display 48 displays data acting on the five senses, such as image, text, and sound, under control of CPU 42. Display 48 is configured with, for example, an LCD (Liquid Crystal Display) or an organic EL (Electro-Luminescence) display. CPU 42 executes the movement ability evaluating program to display measurement data transmitted from communication unit 40, data indicating the evaluation result of the movement ability, and data indicating exercise advice, on display 48. CPU 42 can store these data in internal storage device 68.

(Functional Configuration of Acceleration Sensor 1)

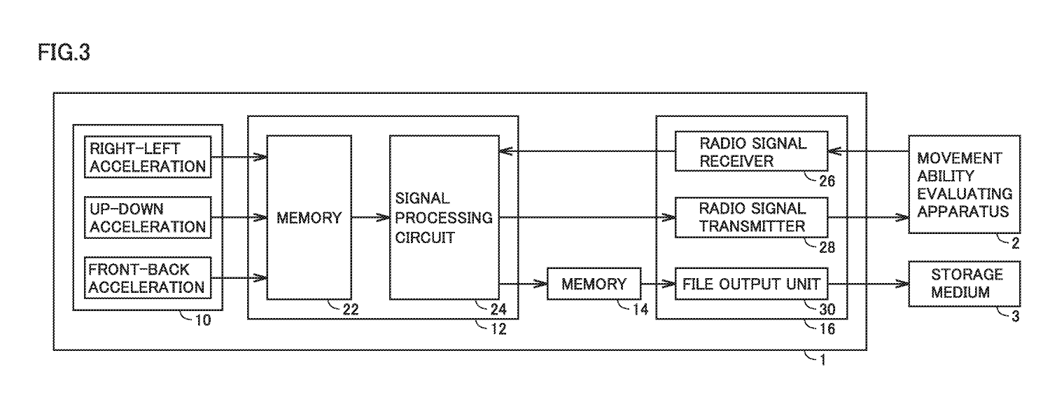

FIG. 3 is a diagram schematically showing a functional configuration of acceleration sensor 1 according to the first embodiment. As shown in FIG. 3, acceleration sensor 1 includes a memory 22 and a signal processing circuit 24. Memory 22 is configured with a storage device such as RAM to store a program, measurement data, and the like.

Signal processing circuit 24 controls each unit in acceleration sensor 1. Signal processing circuit 24 operates under instructions of a program stored in memory 22 and executes a variety of operations including movement ability evaluation described later.

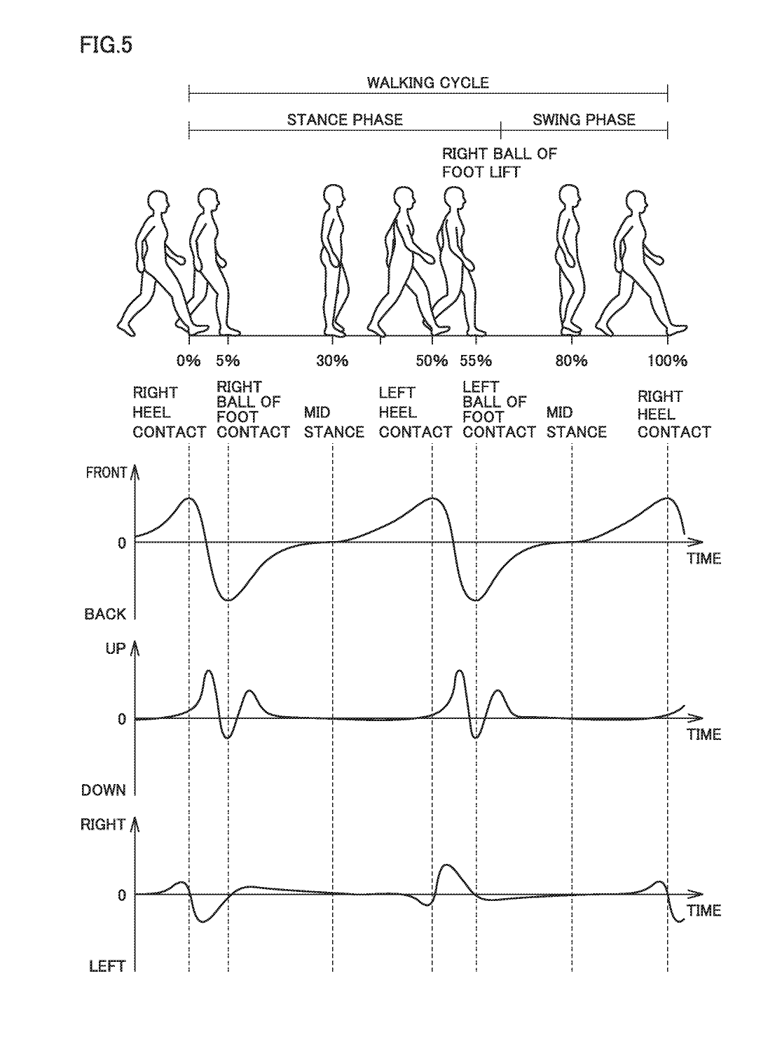

Specifically, signal processing circuit 24 includes a filter for removing noise and an A/D (Analog/Digital) converter and removes noise from an electrical signal output from sensor unit 10 to generate an acceleration signal indicating acceleration as shown in FIG. 5. Signal processing circuit 24 samples the generated acceleration signal at predetermined intervals to generate measurement data.

The sampling interval in signal processing circuit 24 is preferably 1 ms to 200 ms. If the sampling interval is shorter than 1 ms, the computation load in signal processing circuit 24 increases and memory 22 requires a large capacity for storing measurement data. If the sampling interval is longer than 200 ms, it is difficult to accurately grasp change in position of the subject's body center of gravity involved with movement More preferably, the sampling interval in signal processing circuit 24 is about 5 ms. Signal processing circuit 24 outputs the generated measurement data to communication unit 16. The lower limit of the sampling interval is preferably 2 ms or more, more preferably 5 ms or more. The upper limit of the sampling interval is preferably 100 ms or less, more preferably 50 ms or less, further preferably 20 ms or less.

Communication unit 16 includes a radio signal receiver 26, a radio signal transmitter 28, and a file output unit 30. Radio signal receiver 26 receives an operation instruction from movement ability evaluating apparatus 2 and applies the received operation instruction to signal processing circuit 24. The operation instruction includes an instruction for specifying a destination to save measurement data generated by signal processing circuit 24.

Radio signal transmitter 28 transmits the measurement data generated by signal processing circuit 24 to movement ability evaluating apparatus 2. Movement ability evaluating apparatus 2 receives the measurement data transmitted from radio signal transmitter 28 and stores the measurement data into internal storage device 68 (see FIG. 4).

Signal processing circuit 24 stores the generated measurement data into memory 14. Signal processing circuit 24 is configured to select one of internal memory 14 of acceleration sensor 1 and a storage device (storage device 68 in movement ability evaluating apparatus 2) external to acceleration sensor 1 in accordance with an operation instruction from movement ability evaluating apparatus 2 (or predetermined setting) to save the measurement data.

In this manner, when the movement ability is evaluated using acceleration sensor 1, signal processing circuit 24 can transmit measurement data by sensor unit 10 in real time to movement ability evaluating apparatus 2 through radio signal transmitter 28. Therefore, movement ability evaluating apparatus 2 can evaluate the movement ability of subject M in real time, based on the received measurement data.

Alternatively, signal processing circuit 24 may store the measurement data in memory 14. File output unit 30 can transmit the measurement data stored in memory 14 to external storage medium 3. For example, a USB memory and a memory stick (registered trademark) can be used as external storage medium 3.

With this configuration, even in a situation in which wireless communication between acceleration sensor 1 and movement ability evaluating apparatus 2 is difficult, acceleration sensor 1 stores the measurement data in memory 14 so that the measurement data stored in memory 14 can be read via storage medium 3 later to evaluate the movement ability. Alternatively, the acceleration produced at the waist of subject M is measured over a few hours (or a few days), and the measurement data is stored in memory 14 so that the exercise habit of subject M can be evaluated in addition to the movement ability of subject M, based on the measurement data read from storage medium 3. Acceleration sensor 1 may be configured to read measurement data via wired data transmission means such as USB, rather than via storage medium 3.

(Functional Configuration of Movement Ability Evaluating Apparatus 2)

FIG. 4 is a diagram schematically showing a functional configuration of movement ability evaluating apparatus 2 according to the first embodiment.

As shown in FIG. 4, in movement ability evaluating apparatus 2, communication unit 40 includes a radio signal receiver 60 and a radio signal transmitter 62. Radio signal receiver 60 receives measurement data from acceleration sensor 1 and transmits the received measurement data to CPU 42.

CPU 42 includes a control unit 64 and a storage device 68. Storage device 68 includes, for example, a ROM (Read Only Memory) and a RAM. The ROM stores a program for controlling movement ability evaluating apparatus 2. The program includes a movement ability evaluating program. The RAM stores data for setting a variety of functions of movement ability evaluating apparatus 2, measurement data, data indicating the evaluation result of the movement ability, and data indicating exercise advice.

Control unit 64 is configured with a processor. Control unit 64 operates under instructions of a program stored in storage device 68 to control the operation of movement ability evaluating apparatus 2. Control unit 64 operates under instructions of the movement ability evaluating program to fulfill the functions as an evaluation unit 70 and a determination unit 72.

Evaluation unit 70 evaluates the movement ability of subject M, based on the measurement data acquired by radio signal receiver 60. Alternatively, evaluation unit 70 evaluates the movement ability of subject M, based on the measurement data read from storage medium 3. As described above, the movement ability at least includes front-back balance, right-left balance, and weight shift. In the present embodiment, in total, six items including these three items plus muscle strength, walking speed, and rhythm are evaluated. These items are not essential and may include items other than these items.

Evaluation unit 70 calculates an indicator indicating the movement ability of subject M, based on the measurement data. Evaluation unit 70 gives a score to the calculated indicator, for example, where the ideal value is 10 points (maximum). In this way, the movement ability of subject M is quantitatively evaluated by giving a score to each indicator. This enables the user to quantitatively grasp which of the six items is inferior.

Determination unit 72 acquires the evaluation result from evaluation unit 70 and accepts external data input by the user from operation accepting unit 50. The external data includes subject identification information that is information for identifying subject M and a data threshold list. The subject identification information includes name, gender, age, height, and weight of subject M. The data threshold list is data of thresholds for use in determining exercise advice. Determination unit 72 refers to the data threshold list to determine exercise advice suitable for subject M, based on the evaluation result of the movement ability of subject M.

Control unit 64 displays measurement data, the evaluation result by evaluation unit 70, and data indicating the exercise advice by determination unit 72, on display 48. Control unit 64 stores these data into storage device 68.

(Operation of Movement Ability Evaluating System 100)

The operation of movement ability evaluating system 100 according to the first embodiment will now be described in detail.

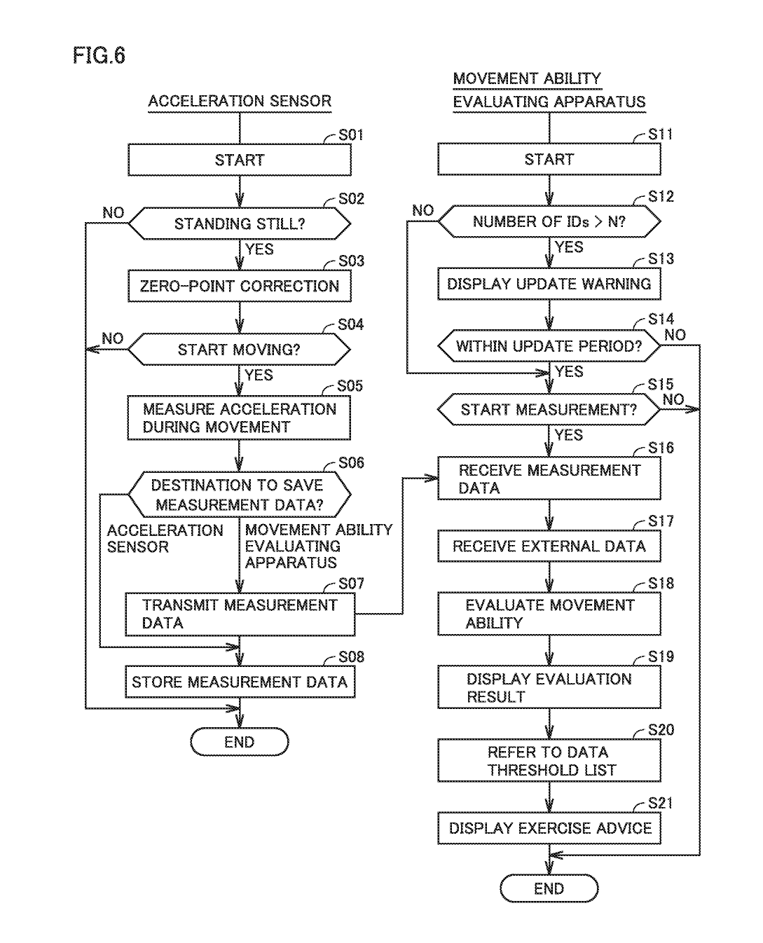

FIG. 5 shows the relation between a human walking cycle and front-back acceleration, up-down acceleration and right-left acceleration during walking. As shown in FIG. 5, a human walking cycle refers to the time from when the heel of one foot (right leg in FIG. 6) touches the ground to when the heel of this foot (right leg) touches the ground next time. The foot in contact with the ground to support the weight is referred to as “supporting leg”, and the foot lifting off the ground and swinging forward is referred to as “idling leg”. The walking cycle includes a “stance phase” with the foot on the ground and a “swing phase” with the foot off the ground.

The stance phase starts with a state in which the heel of the foot serving as the idling leg is in contact with the ground (heel contact), followed by a state in which the ball of the foot comes into contact with the ground and the entire sole touches the ground (ball of foot contact), a state in which the weight is supported only by the supporting leg and the body is upright (mid stance), and a state from the sole in contact with the ground to the heel off the ground (heel lift), and ends with a state in which the ball of foot lifts off the ground whereby the foot lifts off the ground (ball of foot lift). That is, in each of the right and left feet, the time from heel contact to ball of foot lift is the stance phase, and the time from ball of foot lift to heel contact is the swing phase.

During human walking, the human body center of gravity shifts in the front-back direction, the right-left direction, and the up-down direction. FIG. 5 shows exemplary temporal waveforms of front-back acceleration, up-down acceleration, and right-left acceleration in one walking cycle when a person is walking on a level ground. As shown in FIG. 5, during walking, the right and left feet alternately serve as supporting leg, so that a periodicity appears in the temporal waveforms of accelerations in the front-back direction, the right-left direction, and the up-down direction. In the temporal waveforms of acceleration shown in FIG. 5 and subsequent figures, the forward direction, the upward direction, and the right direction are positive direction. However, the backward direction, the downward direction, and the left direction may be positive direction.

In the present embodiment, for each of the right and left feet, an indicator indicating the movement ability of subject M is calculated based on the temporal waveforms of acceleration in a period of time mainly from heel contact to mid stance in the stance phase. This is because there is a deviation in shift of the body center of gravity in at least one of the front-back direction, the right-left direction, and the up-down direction in a period of time from heel contact to mid stance when the motor function decreases because of aging, motor disorder, etc.

When the movement ability is evaluated by movement ability evaluating system 100, first of all, with acceleration sensor 1 attached to the waist of subject M, the power switch of each of acceleration sensor 1 and movement ability evaluating apparatus 2 is turned on to start acceleration sensor 1 and movement ability evaluating apparatus 2.

Movement ability evaluating apparatus 2 accepts input operation indicating an instruction to start evaluation through operation accepting unit 50 and then instructs acceleration sensor 1 to start measurement through communication unit 40. Acceleration sensor 1 corrects the measured value of sensor unit 10 when subject M is standing still to a zero point of front-back acceleration, right-left acceleration, and up-down acceleration. The front-back acceleration, right-left acceleration, and up-down acceleration produced during movement of the subject thus can be precisely measured.

Subject M moves barefoot straight forward by a predetermined distance. In the present embodiment, it is assumed that subject M moves at a speed of 0.5 km to 5 km per hour. When it is determined that subject M starts moving, acceleration sensor 1 measures front-back acceleration, right-left acceleration, and up-down acceleration during movement of subject M and outputs measurement data to movement ability evaluating apparatus 2 through communication unit 16. Movement ability evaluating apparatus 2 acquires measurement data from a signal output by acceleration sensor 1.

FIG. 6 is a flowchart for explaining movement ability evaluation executed by movement ability evaluating system 100 according to the first embodiment. Movement ability evaluating apparatus 2 executes the movement ability evaluating program to communicate with acceleration sensor 1 by radio and execute the process shown in FIG. 6. The process in the flowchart shown in FIG. 6 is executed, for example, at certain time intervals.

Referring to FIG. 2 to FIG. 4 and FIG. 6, in acceleration sensor 1, at step S01, power supply 20 is turned on to start acceleration sensor 1 attached to the waist of subject M. Then, at step S02, signal processing circuit 24 determines whether subject M is standing still, based on an output signal of sensor unit 10. Specifically, if there is no significant change in each of front-back acceleration, right-left acceleration, and up-down acceleration (for example, if the variation range of each acceleration falls below a threshold), signal processing circuit 24 determines that subject M is standing still.

If it is determined that subject M is standing still (YES in the determination at S02), signal processing circuit 24 proceeds to step S03 and corrects the measured value of sensor unit 10 when subject M is standing still to a zero point of right-left acceleration, up-down acceleration, and front-back acceleration. On the other hand, if subject M is not standing still (NO in the determination at S02), that is, if the subject M is moving, the process ends.

At step S04, signal processing circuit 24 determines whether subject M starts moving, based on an output signal from sensor unit 10. If a change is observed in at least one of front-back acceleration, right-left acceleration, and up-down acceleration (for example, if the variation range of at least one acceleration is greater than a threshold), signal processing circuit 24 determines that subject M starts moving.

If subject M starts moving (YES in the determination at S04), at step S05, signal processing circuit 24 measures up-down acceleration, right-left acceleration, and front-back acceleration produced at the waist of subject M. Signal processing circuit 24 converts an acceleration signal output by sensor unit 10 into measurement data. On the other hand, if subject M does not start moving (NO in the determination at S04), the process ends.

At step S06, signal processing circuit 24 determines which of storage device 68 of movement ability evaluating apparatus 2 and memory 14 of acceleration sensor 1 is specified as a destination to save the measurement data. If the destination to save the measurement data is storage device 68, signal processing circuit 24 proceeds to step S07 and transmits the measurement data to movement ability evaluating apparatus 2 through communication unit 16 (radio signal transmitter 28).

On the other hand, if the destination to save the measurement data is memory 14, signal processing circuit 24 proceeds to step S08 and stores the measurement data into memory 14.

In movement ability evaluating apparatus 2, when power supply 46 is turned on to start at step S1, at step S12, control unit 64 determines whether the number of IDs already issued for the subject registered in movement ability evaluating apparatus 2 exceeds a maximum permissible number N set for the same account. If the number of IDs issued exceeds the maximum permissible number N (YES in the determination at S12), control unit 64 proceeds to step S13 and produces a warning to prompt for an update process for changing (increasing) the maximum permissible number. The warning is given, for example, by displaying a message on display 48 to prompt for an update process or by reading the message by voice.

At step S14, control unit 64 determines whether the present time is within an update period for the maximum permissible number of the number of IDs. If it is determined that the present time is within an update period (YES in the determination at S14), control unit 64 permits execution of the process of evaluating the movement ability of subject M. If it is determined that the present time is not within an update period (NO in the determination at S14), the process ends.

At step S15, control unit 64 determines whether an input operation indicating an instruction to start measurement is accepted by operation accepting unit 50. If an input operation indicating an instruction to start measurement is accepted (YES in the determination at S15), at step S16, communication unit 40 receives measurement data of acceleration sensor 1. The received measurement data is sent to control unit 64.

At step S17, communication unit 40 further receives external data. The external data includes subject identification information that is information for identifying subject M and a data threshold list. The subject identification information includes information such as name, gender, age, height, and weight of subject M. The data threshold list is used to determine exercise advice suitable for subject M in accordance with the evaluation result of movement ability, as will be described later.

At step S18, control unit 64 evaluates the movement ability of subject M, based on the measurement data transmitted from acceleration sensor 1. Specifically, control unit 64 calculates an indicator indicating the movement ability of subject M, based on the temporal waveform of acceleration measured during movement of subject M.

At step S19, control unit 64 displays the evaluation result of the movement ability on display 48. A display example of the evaluation result on display 48 will be described in detail later.

At step S20, control unit 64 refers to the data threshold list to determine exercise advice suitable for subject M, based on the evaluation result. In the data threshold list, a plurality of thresholds classified according to age, gender, and the like are registered for each indicator. Control unit 64 refers to the data threshold list to set a threshold appropriate for subject M, based on the subject identification information.

Subsequently, control unit 64 compares the score of the indicator calculated at step S18 with the set threshold to determine whether the movement ability of subject M decreases. For example, if the indicator indicating front-back balance is lower than the threshold, control unit 64 determines that the front-back balance ability decreases. Control unit 64 further determines the degree of decrease in the front-back balance ability, based on the difference between the indicator and the threshold.

Control unit 64 then determines exercise advice for improving the front-back balance ability of subject M, in accordance with the degree of decrease in the front-back balance ability.

At step S21, control unit 64 displays the determined exercise advice on display 48. A display example of exercise advice on display 48 will be described in detail later.

The evaluation result at step S18 and the exercise advice at step S20 are provided to the user on display 48 and stored into storage device 68 of movement ability evaluating apparatus 2 in association with the measurement data of subject M.

(Movement Ability Evaluation)

The process of evaluating the movement ability of subject M based on measurement data will now be described.

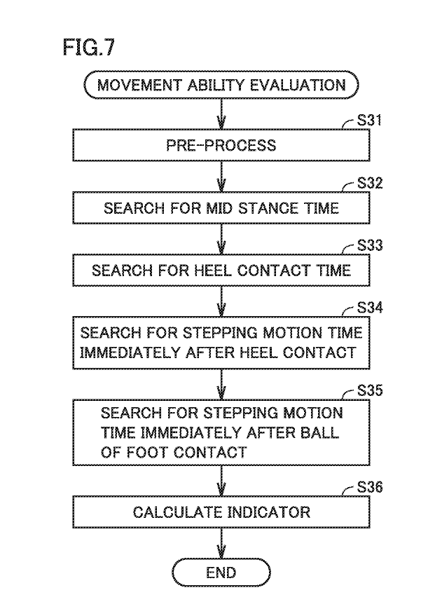

FIG. 7 is a flowchart for explaining the procedure of evaluating the movement ability at step S18 in FIG. 6. As shown in FIG. 7, at step S31, control unit 64 executes a pre-process for calculating an indicator indicating the movement ability from the measurement data. Control unit 64 then searches for the time when a certain operation is performed in the temporal waveform (see FIG. 6) of three-axis acceleration that is the measurement data. Control unit 64 searches for a mid stance time (S32), searches for a heel contact time (S33), searches for a stepping motion time immediately after heel contact (S34), and searches for a stepping motion time immediately after ball of foot contact (S35). Subsequently, at step S36, control unit 64 calculates an indicator indicating the movement ability of subject M, based on the temporal waveform of acceleration in a period of time specified by the found times.

The detailed operation at each of S31 to S36 shown in FIG. 7 will be described below.

(S31: Pre-process)

At step S31, control unit 64 performs smoothing processing for temporal waveforms of front-back acceleration, right-left acceleration, and up-down acceleration. This processing attenuates a high frequency component included in the temporal waveform of acceleration. Control unit 64 first-differentiates the temporal waveform of acceleration subjected to smoothing processing to generate a first derivative waveform of acceleration.

(S32: Search for Mid Stance Time)

Next, control unit 64 searches for the time (mid stance time) Ms when mid stance is performed, from the temporal waveform of acceleration subjected to the pre-process, for each of the right and left legs. In searching, a search range to be searched for mid stance time Ms is initially set. The temporal waveform and the first derivative waveform of front-back acceleration are used for setting the search range.

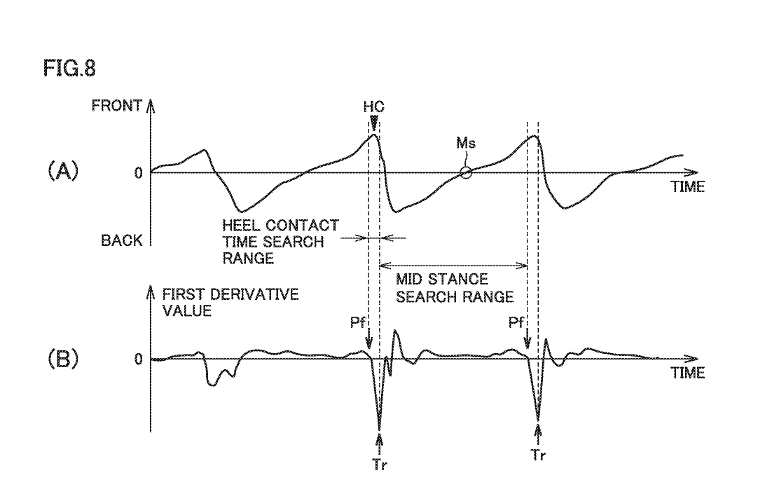

FIG. 8(A) shows an example of the temporal waveform of front-back acceleration measured during movement of subject M. FIG. 8(B) shows the first derivative waveform of the front-back acceleration shown in FIG. 8(A). Referring to FIG. 8B, a plurality of deep grooves (hereinafter referred to as troughs) Tr appear in the first derivative waveform of front-back acceleration. Each of troughs Tr corresponds to an inflection point at which front-back acceleration turns from the forward direction to the backward direction.

At step S32, first of all, trough Tr is found in the first derivative waveform of front-back acceleration, and then peak Pf to the immediate left closest to this trough Tr is found. That is, peak Pf immediately before trough Tr is found. Then, the time range from the position of any one trough Tr to the position of peak Pf immediately before trough Tr next to this trough Tr is set as a search range for mid stance time Ms.

Next, mid stance time Ms is searched for within the set search range. Specifically, referring to FIG. 8A, the time when the absolute value of front-back acceleration is smallest is searched for in the search range. In the example in FIG. 8 A, the time when the absolute value of front-back acceleration is smallest corresponds to the time when front-back acceleration is zero (zero cross time).

(S33: Search for Heel Contact Time) At step S33, control unit 64 searches for the time (heel contact time) HC when heel contact is performed, from the temporal waveform of acceleration, for each of the right and left legs. In searching, the search range to be searched for heel contact time HC is set. In setting the search range, the temporal waveform and the first derivative waveform of front-back acceleration are used.

Referring to FIG. 8(B), in the first derivative waveform of front-back acceleration, trough Tr and peak Pf to the immediate left closest to the position of this trough Tr (That is, the peak immediately before this trough Tr) are found. Then, the time range from the position at any one trough Tr to the position of peak Pf immediately before this trough Tr is set as a search range of heel contact time.

Next, control unit 64 searches for heel contact time HC in the set search range. Since the body center of gravity decelerates in the backward direction as a result of the heel touching the ground during walking, front-back acceleration exhibits an inflection point from the forward direction to the backward direction. Then, in the temporal waveform of front-back acceleration shown in FIG. 8(A), the search range is searched for the time when the inflection point from the forward direction to the backward direction appears, that is, the time when front-back acceleration is largest.

(S34: Search for Stepping Motion Time Immediately after Heel Contact)

At step S34, control unit 64 searches for the time (stepping motion time immediately after heel contact) T1 when stepping motion is performed immediately after heel contact, from the temporal waveform of acceleration, for each of the right and left feet. In searching, a search range to be searched for stepping motion time T1 immediately after heel contact is set. In setting a search range, the temporal waveform of up-down acceleration and the first derivative waveform of front-back acceleration are used.

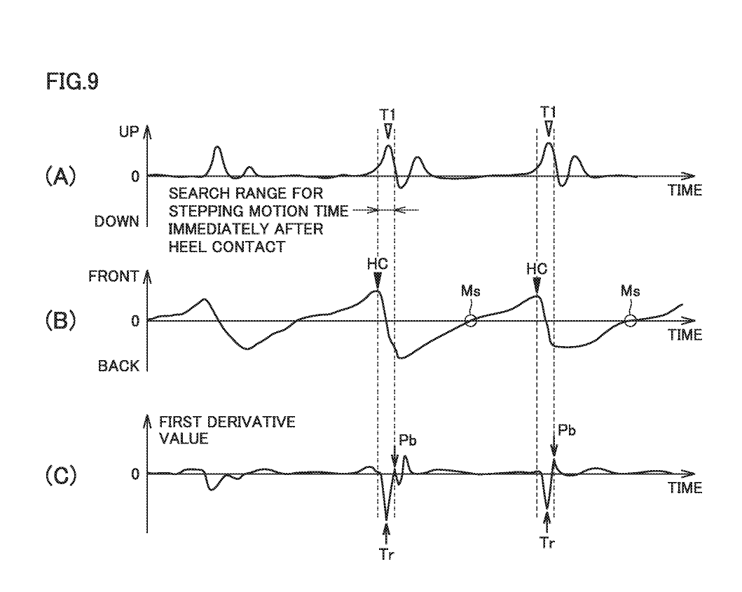

FIG. 9(A) shows an example of the temporal waveform of up-down acceleration measured during movement of subject M. FIG. 9(B) shows an example of the temporal waveform of front-back acceleration measured during movement of subject M. FIG. 9(C) shows the first derivative waveform of front-back acceleration shown in FIG. 9(B). At step S34, peak Pb to the immediate right closest to the position of trough Tr is found in the first derivative waveform of front-back acceleration. That is, peak Pb immediately after trough Tr is found. Peak Pb corresponds to that the body center of gravity decelerating in the backward direction due to heel contact is received (that is, the body center of gravity is pulled back in the forward direction). The time range from the position of any one trough Tr to the position of peak Pb immediately after this trough Tr is set as a search range for stepping motion time T1 immediately after heel contact.

Next, control unit 64 searches for stepping motion time T1 immediately after heel contact in the set search range. Since the body center of gravity during walking ascends as a result of stepping immediately after heel contact, the up-down acceleration exhibits an inflection point from the upward direction to the downward direction immediately after heel contact time HC. Then, in the temporal waveform of up-down acceleration shown in FIG. 9(A), the time when an inflection point from the upward direction to the downward direction appears, that is, the time when up-down acceleration is largest is searched for in the search period.

(S35: Search for Stepping Motion Time Immediately after Ball of Foot Contact)

At step S35, control unit 64 searches for the time (stepping motion time immediately after ball of foot contact) T2 when stepping motion is performed immediately after the ball of the foot comes into contact with the ground, from the temporal waveform of acceleration, for each of the right and left feet. In searching, a search range to be searched for stepping motion time T2 immediately after ball of foot contact is set. In setting a search range, the temporal waveform of up-down acceleration and the first derivative waveform of up-down acceleration are used.

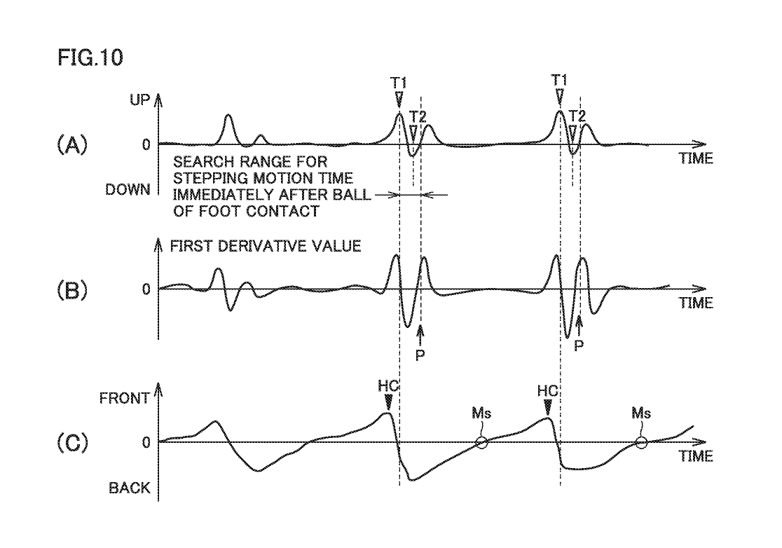

FIG. 10(A) shows an example of the temporal waveform of up-down acceleration measured during movement of subject M. FIG. 10(B) shows the first derivative waveform of up-down acceleration shown in FIG. 10(A). FIG. 10(C) shows an example of the temporal waveform of front-back acceleration measured during movement of subject M. At step S35, in the first derivative waveform of up-down acceleration, peak P is found in a time range from stepping motion time T1 immediately after heel contact to mid stance time Ms. Peak P corresponds to that the body center of gravity during walking ascends as a result of stepping with the ball of the foot. The time range from stepping time T1 immediately after heel contact to the position of peak P is set as a search range for stepping motion time T2 immediately after ball of foot contact.

Next, control unit 64 searches for stepping motion time T2 immediately after ball of foot contact in the set search range. The body center of gravity during walking ascends as a result of stepping on the ground with the heel, then descends as a result of the ball of the foot touching the ground, and ascends again as a result of stepping on the ground with the ball of the foot. Therefore, the up-down acceleration exhibits an inflection point from the downward direction to the upward direction immediately after stepping motion time TI immediately after heel contact. Then, in the temporal waveform of up-down acceleration shown in FIG. 10(A), the time when an inflection point from the downward direction to the upward direction appears, that is, the time when up-down acceleration is smallest is searched for in the search period.

(S36: Calculation of Indicator)

At step S36, control unit 64 calculates an indicator indicating the movement ability of subject M, based on the temporal waveform of acceleration in a period of time from the found heel contact time HC to mid stance time Ms.

A method of calculating an indicator indicating each of front-back balance, weight shift, and right-left balance will be described below.

(1) Front-Back Balance

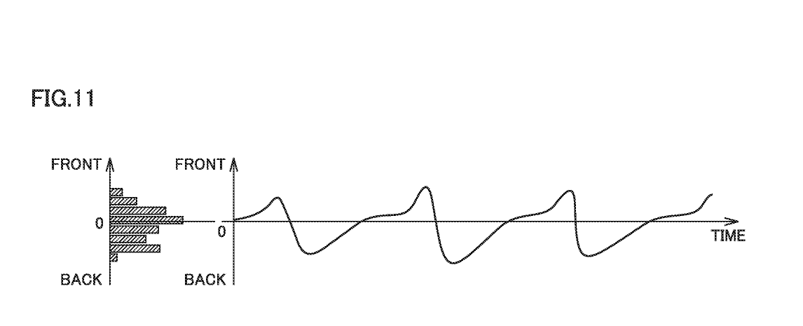

FIG. 11 shows an example of the temporal waveform of front-back acceleration measured during movement of subject M. Control unit 64 calculates an indicator indicating front-back balance, based on a distribution state of forward acceleration and backward acceleration in a temporal waveform of front-back acceleration in at least one walking cycle.

FIG. 11 shows a histogram of front-back acceleration in a plurality of walking cycles that is generated based on a temporal waveform of front-back acceleration. In this histogram, the lateral axis (the axis extending vertically in the figure) shows front-back acceleration, and the longitudinal axis (the axis extending horizontally in the figure) shows frequency.

When subject M is moving in a correct posture, the distribution is almost equal between forward acceleration and backward acceleration in the histogram. The distribution almost equal means that the distribution of forward acceleration and the distribution of backward acceleration are in line symmetry.

By contrast, when subject M is in a lean forward posture, the body center of gravity is inclined forward and therefore the frequency of forward acceleration tends to be greater than the frequency of backward acceleration in the histogram. On the other hand, when subject M is in a lean backward posture, the body center of gravity is inclined backward and therefore the frequency of backward acceleration tends to be greater than the frequency of forward acceleration in the histogram.

For the histogram, control unit 64 calculates the total value ΣAF by summing the frequencies of forward acceleration and calculates the total value ΣAB by summing the frequencies of backward acceleration.

When subject M is moving in a correct posture, the total value ΣAF and the total value ΣAB are equal and the ratio ΣAF/ΣAB is close to 1. In the present description, two values being equal is defined as the concept that includes both of the case where two values agree and the case where two values do not perfectly match but their difference is sufficiently small.

By contrast, in the case of a lean forward posture, the total value ΣAF is greater and the ratio ΣAF/ΣAB is a value greater than 1. On the other hand, in the case of a lean backward posture, the total value ΣAB is greater and the ratio ΣAF/ΣAB is smaller than 1. Control unit 64 gives a score to the calculated ΣAF/ΣAB, where ΣAF/ΣAB=1 is the ideal value (10 points).

(2) Weight Shift

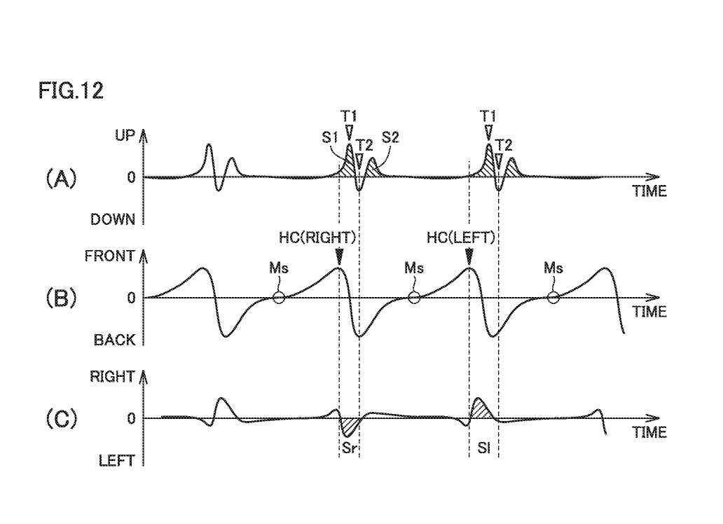

FIGS. 12(A) to 12(C) show an example of temporal waveforms of up-down acceleration, front-back acceleration, and right-left acceleration measured during movement of subject M. For one foot, control unit 64 calculates an indicator of weight shift of the sole of the one foot, based on the temporal waveform of up-down acceleration in a period of time from heel contact time HC to mid stance time Ms.

As shown in FIG. 12(A), in a period of time from heel contact time HC to mid stance time Ms, two peaks appear in the temporal waveform of up-down acceleration. The first peak appears at stepping motion time T1 immediately after heel contact. The second peak appears immediately after stepping motion time T2 immediately after ball of foot contact. This is because the body center of gravity ascends as a result of stepping on the ground with the heel immediately after the heel touches the ground, then the body center of gravity descends as a result of the ball of the foot touching the ground, and the body center of gravity ascends again as a result of stepping on the ground with the ball of the foot.

However, when the motor function such as muscle strength decreases, the motion of stepping on the ground with the ball of the foot may be difficult. Consequently, in the temporal waveform of up-down acceleration, the height of the second peak is lower or no second peak appears.

Control unit 64 calculates a value of integral S1 by time-integrating the upward acceleration in a period of time from heel contact time HC to stepping motion time T2 immediately after ball of foot contact. Control unit 64 further calculates a value of integral S2 by time-integrating the upward acceleration in a period of time from stepping motion time T2 immediately after ball of foot contact to mid stance time Ms Control unit 64 then calculates an indicator indicating weight shift, based on the ratio between the value of integral S1 and the value of integral S2 (S2/S1).

When the motor function is normal, the value of integral S1 and the value of integral S2 are equal and therefore the ratio S2/S1 is a value close to 1. However, when the motor function decreases, the second peak is lower or disappears as described above, and the value of integral S2 is smaller. Consequently, the ratio S2/S1 is a value smaller than the value when the motor function is normal. Control unit 64 gives a score to the calculated ratio S2/S1, where the ratio S2/S1=1 is an ideal value (10 points).

(3) Right-Left Balance

As shown in FIG. 12(C), a peak appears immediately after heel contact time HC in a temporal waveform of right-left acceleration. This is because the body center of gravity during walking shifts in the left direction as a result of the right heel touching the ground and shifts in the right direction as a result of the left heel touching the ground. That is, a peak appears in the left direction immediately after the time IC (hereinafter referred to as right heel contact time) when the right heel touches the ground, and a peak appears in the right direction immediately after the time HC (hereinafter referred to as left heel contact time) when the left heel touches the ground.

When the subject is moving in a correct posture, the peak in the left direction and the peak in the right direction are equal in height. On the other hand, when the physical function decreases to cause posture imbalance, the body center of gravity is deviated either to the right or to the left, so that the peak of one of the right and left directions is lower than the peak of the other. That is, the peaks are unequal between the right and the left.

Control unit 64 calculates an indicator indicating right-left balance, based on a temporal waveform of leftward acceleration in a period of time from right heel contact time HC to right mid stance time Ms and a temporal waveform of rightward acceleration from left heel contact time HC to left mid stance time Ms. Specifically, control unit 64 calculates a value of integral Sr by time-integrating the leftward acceleration in a period of time from right heel contact time HC to right mid stance time Ms. Control unit 64 also calculates a value of integral S1 by time-integrating rightward acceleration in a period of time from left heel contact time HC to left mid stance time Ms. Control unit 64 then calculates an indicator indicating right-left balance, based on the ratio between the value of integral Sr and the value of integral S1 (Sr/Sl).

When the subject is moving in a correct posture, the value of integral Sr is equal to the value of integral Sl and therefore the ratio Sr/Sl is a value close to 1. On the other hand, if the body center of gravity is inclined to the left, the body center of gravity shifts in the left direction when the right heel touches the ground, and the value of integral Sr is greater, so that the ratio Sr/Sl is a value greater than 1. When the body center of gravity is inclined to the right, the body center of gravity shifts in the right direction when the left heel touches the ground, and the value of integral Sl is greater, so that the ratio Sr/Sl is a value smaller than 1. Control unit 64 gives a score to the calculated ratio Sr/Sl, where the ratio Sr/Sl=1 is an ideal value (10 points).

(Display Example on Display 48)

A display example on display 48 in movement ability evaluating apparatus 2 will now be described.

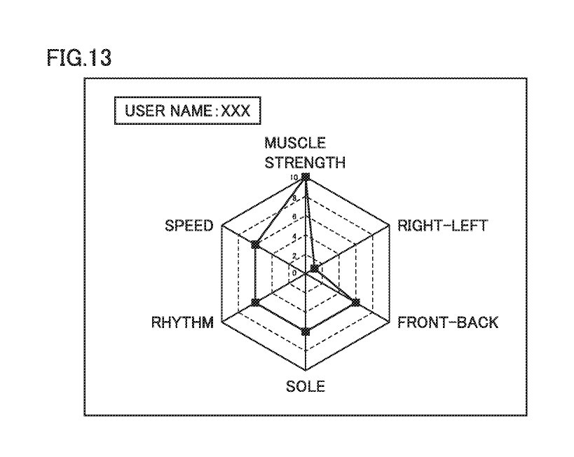

FIG. 13 is a diagram showing an example of the screen that displays the result of evaluation of the movement ability of subject M by control unit 64 on display 48.

As shown in FIG. 13, identification information of subject M who logs in to movement ability evaluating apparatus 2 appears on the screen of display 48. For example, the name “XXX” of subject M is displayed.

Display 48 further displays the evaluation result of the movement ability of subject M. In the example in FIG. 13, the evaluation result of the movement ability of subject M is displayed in a graph in the form of a radar chart. The graph has six items: muscle strength, right-left, front-back, sole, rhythm, and speed, as items of movement ability. The “right-left” indicates right-left balance, “front-back” indicates front-back balance, and “sole” indicates weight shift “Muscle strength” indicates the magnitude and the state of motion at least including lower limb muscle strength, “rhythm” indicates walking pace, and “speed” indicates walking speed.

The graph shows a score for each item, where 10 points is the ideal value. This allows the user or subject M to view the screen on display 48 and learn which item is inferior by what degree in a quantitative manner.

The graph appearing on display 48 is preferably in such a format that provides intuitive understanding of a score for each item. For example, the graph may be a bar graph illustrating a score for each item.

Although not illustrated in the figure, a graph showing the evaluation result in the past may be displayed in addition to the graph in FIG. 13. In this manner, the user can learn which item decreases by what degree and which item improves by what degree compared with the evaluation in the past, in a quantitative manner. Alternatively, the target value or the mean value for the age of subject M may be displayed together in the graph in FIG. 13. In addition, the target value or the average value of population having common features for at least pan of external data (age, gender, etc.) may be displayed together. In this manner, the user can learn which item is inferior to the target value or the mean value by what degree, in a quantitative manner. Such display provides subject M motivation to improve the movement ability.

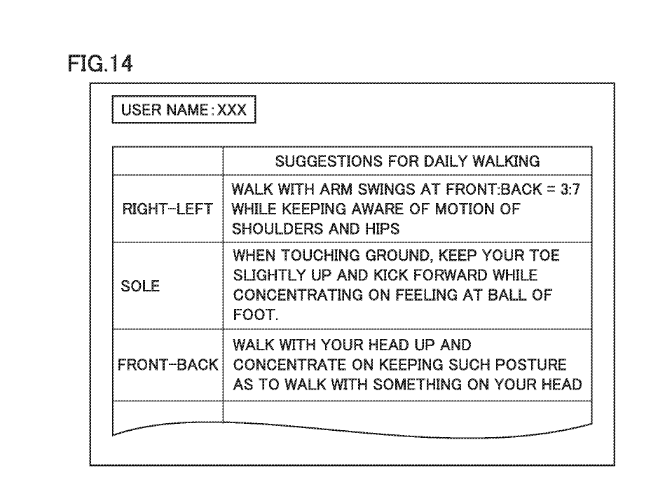

FIG. 14 is a diagram showing an example of the screen that displays exercise advice determined by control unit 64 based on the evaluation result on display 48.

As shown in FIG. 14, identification information (for example, the name of subject M) of subject M who logs in to movement ability evaluating apparatus 2 as well as exercise advice suitable for subject M appears on the screen of display 48.

Exercise advice corresponding to the evaluated movement ability appears on display 48. In the example in FIG. 14, exercise advice determined based on the evaluation result shown in FIG. 13 is illustrated. In FIG. 14, suggestions for usual walking are displayed in text for each item as exercise advice. The suggestions may be depicted using pictures. This prompts subject M to pay attention so as to move in a correct posture and a correct weight shift.

According to the first embodiment, the movement ability of a subject can be properly evaluated by using at least one of front-back balance, right-left balance, and weight shift of the subject during movement, as an indicator for evaluating the movement ability of the subject. The risk of falling of the subject thus can be determined precisely.

### Second Embodiment

In the first embodiment, the time when a certain motion such as mid stance, heel contact, and ball of foot contact is performed is searched for in the temporal waveform of acceleration measured by acceleration sensor 1, and an indicator indicating the movement ability of subject M is calculated based on the temporal waveform of acceleration in a period of time specified by the found time. However, an indicator may be calculated without searching for the time of a certain operation.

In a second embodiment, an indicator indicating the movement ability of subject M is calculated based on an autocorrelation function of acceleration, as an example. The configuration of the movement ability evaluating system according to the second embodiment is the same as the configuration of movement ability evaluating system 100 according to the first embodiment shown in FIG. 1 to FIG. 4 and will not be further elaborated. The operation of movement ability evaluating apparatus 2 according to the second embodiment will now be described below.

(Operation of Movement Ability Evaluating System 100)

Movement ability evaluating system 100 according to the second embodiment basically executes the movement ability evaluating process shown in FIG. 6. The movement ability evaluating process according to the second embodiment differs from the movement ability evaluating process according to the first embodiment in the procedure of evaluating the movement ability at step S18.

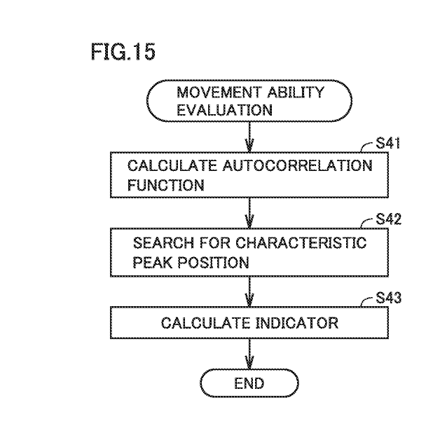

FIG. 15 is a flowchart for explaining the procedure of evaluating the movement ability at step S18 in FIG. 6.

Referring to FIG. 15, at step S41, control unit 64 of movement ability evaluating apparatus 2 calculates an autocorrelation function for each of right-left acceleration, up-down acceleration, and front-back acceleration. In the following description, the autocorrelation function of right-left acceleration is denoted as ACF_X, the autocorrelation function of up-down acceleration is denoted as ACF_Y, and the autocorrelation function of front-back acceleration is denoted as ACF_Z.

At step S42, control unit 64 searches for a characteristic peak position for each of autocorrelation functions ACF_X, ACF_Y, ACF_Z.

At step S43, control unit 64 calculates an indicator indicating the movement ability of subject M, using the found peak position.

A method of calculating an indicator indicating each of front-back balance, weight shift, and right-left balance based on the autocorrelation function of acceleration will be described below.

(1) Front-Back Balance

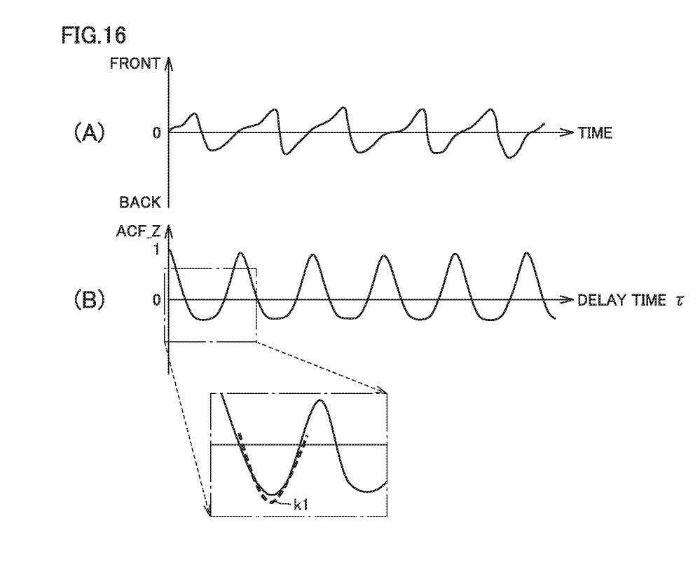

FIG. 16(A) shows an example of the temporal waveform of front-back acceleration measured during movement of subject M FIG. 16(B) shows autocorrelation function ACF_Z(τ) of the front-back acceleration shown in FIG. 16(A), where T is a variable representing delay time.

As shown in FIG. 16(B), with origin (τ=0), peaks periodically appear in autocorrelation function ACF_Z. The distance between adjacent two peaks reflects the periodicity of temporal change of front-back acceleration.

In the walking cycle illustrated in FIG. 5, before and after mid stance, a person is advancing forward only with the foot of the supporting leg. Thus, the temporal waveform of front-back acceleration before and after mid stance reflects instability of balance in a state in which the body center of gravity is supported with one foot.

Specifically, when a smooth weight shift is achieved with one foot, the temporal waveform of front-back acceleration before and after mid stance is smooth. In this case, the valley portion positioned between the origin (r=0) and the first peak position in autocorrelation function ACF_Z can be approximated by a quadric curve (dashed line k1 in the figure).

On the other hand, when the body center of gravity is unsteady with one foot and a smooth weight shift fails to be achieved, the temporal waveform of front-back acceleration before and after mid stance varies. In this case, the valley portion positioned between the origin and the first peak position in autocorrelation function ACF_Z is close to be flat at the bottom. As a result, the valley portion deviates from the quadric curve k1.

Based on such a phenomenon, control unit 64 calculates an indicator indicating front-back balance, based on autocorrelation function ACF_Z of front-back acceleration. Specifically, control unit 64 calculates an indicator indicating front-back balance, based on a deviation of the valley portion positioned between the origin and the first peak position of autocorrelation function ACF_Z from the approximate curve k1 obtained by approximating the valley portion to a quadric curve. For example, control unit 64 extracts a local minimum in the valley portion and a local minimum of the approximate curve k1 and gives a score to the difference between the two local minimums, where the difference with a predetermined value is an ideal value (10 points).

(2) Weight Shift

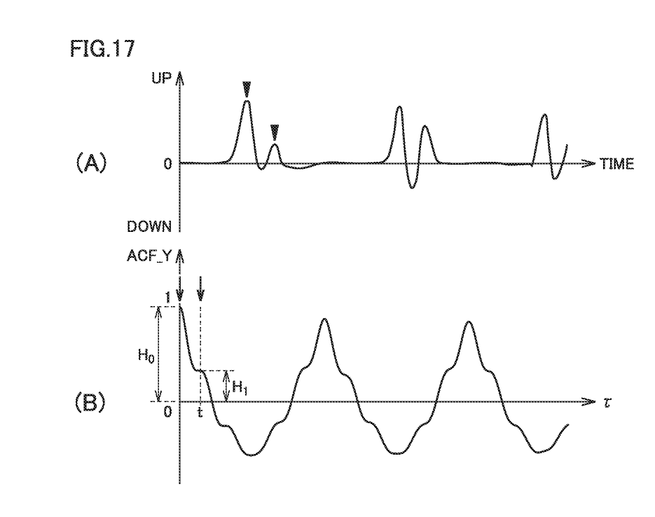

FIG. 17(A) shows an example of the temporal waveform of up-down acceleration measured during movement of subject M. FIG. 17(B) shows autocorrelation function ACF_Y of up-down acceleration shown in FIG. 17(A).

As illustrated in FIG. 12, when the motor function is normal, two peaks (corresponding to black triangles in the figure) appear in the temporal waveform of up-down acceleration in a period of time from heel contact time to mid stance time. The first peak appears at the stepping motion time immediately after heel contact. The second peak appears immediately after the stepping motion time immediately after ball of foot contact.

In autocorrelation function ACF_Y of up-down acceleration, the first peak resulting from the two peaks appears at a delay time (τ=t) sufficiently shorter than the walking cycle. The value of autocorrelation function ACF_Y at the origin (τ=0) is denoted as H0, and the value of autocorrelation function ACF_Y at the first peak position is denoted as H1.

On the other hand, when the motor function decreases, the height of the second peak is lower or no second peak appears in the temporal waveform of up-down acceleration. Thus, the height of the first peak is lower or no first peak appears in autocorrelation function ACF_Y.

Based on such a phenomenon, control unit 64 calculates an indicator indicating weight shift, based on autocorrelation function ACF_Y of up-down acceleration. Specifically, control unit 64 calculates an indicator indicating weight shift, based on the ratio (H1/H0) between the value H0 at the origin of autocorrelation function ACF_Y and the value H1 at the first peak position. When the motor function decreases, H1 is smaller and therefore the ratio H1/H0 is also smaller. Control unit 64 gives a score to the ratio H1/H0, where the ratio H1/H02 when the motor function is normal is the ideal value (10 points).

(3) Right-Left Balance

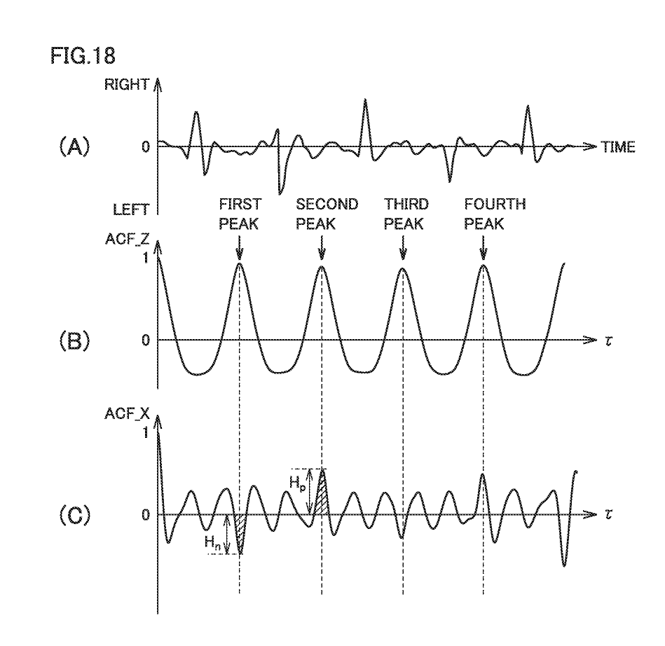

FIG. 18(A) shows an example of the temporal waveform of right-left acceleration measured during movement of subject M. FIG. 18(B) shows autocorrelation function ACF_Z of front-back acceleration shown in FIG. 16(A). FIG. 18(C) shows autocorrelation function ACF_X of right-left acceleration shown in FIG. 18(A).

As illustrated in FIG. 16, autocorrelation function ACF_Z of front-back acceleration reflects the periodicity of temporal change of front-back acceleration, and a plurality of peaks periodically appear.

As shown in FIG. 18(A), in the temporal waveform of right-left acceleration, a peak in the right direction and a peak in the left direction alternately appear for each half cycle of the walking cycle, because the right and left feet alternately serve as the supporting leg. When the subject is moving in a correct posture, the peak in the right direction and the peak in the left direction are equal in height.

As shown in FIG. 18(C), in autocorrelation function ACF_X of right-left acceleration, a peak in the positive direction and a peak in the negative direction alternately appear. When the subject is moving in a correct posture, a peak in the positive direction and a peak in the negative direction alternately appear in autocorrelation function ACF_X at the position equal to the peak position of autocorrelation function ACF_Z. The value of the peak in the positive direction is denoted as Hp, and the value of the peak in the negative direction is denoted as Hn.

Based on such a phenomenon, control unit 64 calculates an indicator indicating right-left balance, based on autocorrelation function ACF_Z of front-back acceleration and autocorrelation function ACF_X of right-left acceleration. Specifically, first of all, control unit 64 searches for the first peak position and the second peak position of autocorrelation function ACF_Z. Next, control unit 64 searches for the value Hn at the peak position corresponding to the first peak position of autocorrelation function ACF_Z, in autocorrelation function ACF_X. Control unit 64 also searches for the value Hp at the peak position corresponding to the second peak position of autocorrelation function ACF_Z, in autocorrelation function ACF_X. Control unit 64 calculates an indicator indicating right-left balance, based on the ratio (|Hp|/|Hn|) between the absolute values of the found value Hn and value Hp.

When the subject is moving in a correct posture, the absolute values of the value Hn and the value Hp are equal and therefore the ratio |Hp/Hn| is a value close to 1. On the other hand, when the body center of gravity is inclined to the left, the value Hn is greater and therefore the ratio |Hp|/|Hn| is a value smaller than 1. When the body center of gravity is inclined to the right, the value Hp is greater and therefore the ratio |Hp/|Hn is a value greater than 1. Control unit 64 gives a score to the calculated ratio |Hp/|Hn, where the ratio |Hp|/|Hn|=1 is the ideal value (10 points).

According to the second embodiment, the movement ability of a subject can be properly evaluated in the same manner as in the first embodiment by using at least one of front-back balance, right-left balance, and weight shift of the subject during movement as an indicator for evaluating the movement ability of subject M. The risk of falling of the subject thus can be determined precisely.

In the second embodiment, the movement ability of the subject can be evaluated by capturing the periodicity of temporal change of acceleration during movement from the autocorrelation function of acceleration. This configuration can reduce the computation processes executed by the control unit of the movement ability evaluating apparatus, compared with the configuration described in the first embodiment in which the movement ability is evaluated by searching for the time when the subject is performing a certain operation in the temporal waveform of acceleration. This achieves fast computation. In other words, while fast computation is achieved, an inexpensive computer can be used, thereby simplifying the system configuration.

<Configuration Example of Movement Ability Evaluating System>

Movement ability evaluating system 100 according to the foregoing first and second embodiments can be implemented using a general computer system rather than a dedicated system. For example, a program (movement ability evaluating program) for executing the movement ability evaluating process described above may be stored in a computer-readable recording medium and distributed so that the program is installed in a computer and the movement ability evaluating process is executed to configure movement ability evaluating system 100. Alternatively, the program may be stored in a server device on a network such as the Internet so that the program can be downloaded in a computer.

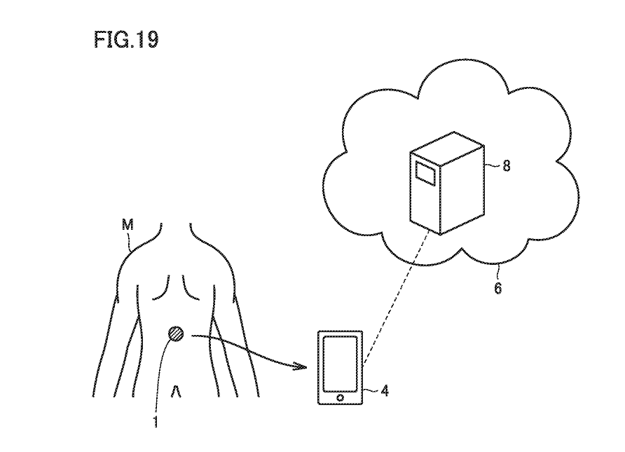

FIG. 19 is a diagram showing another configuration example of movement ability evaluating system 100 according to an aspect of the present invention. As shown in FIG. 19, movement ability evaluating system 100 according to a modification includes an acceleration sensor 1, a communication device 4, and a server 8. Server 8 is connected to a network 6.

Communication device 4 is a terminal used by subject M, for example, a smartphone. Acceleration sensor 1 and communication device 4 communicate with each other by radio. Acceleration sensor 1 and communication device 4 are connected in accordance with short-range wireless communication standards, such as Bluetooth (registered trademark).

Server 8 communicates with communication device 4 to retain measurement data of acceleration sensor 1 as database. Server 8 includes a not-shown memory and control unit. The memory of server 8 is configured with, for example, a flash memory or a RAM and stores a program and various data to be used by server 8. The program includes the movement ability evaluating program. The various data includes data for managing the registered subjects, measurement data acquired for each subject, and a data threshold list.

The control unit of server 8 evaluates the movement ability of a subject, based on the measurement data of the subject stored in the memory and transmits the evaluation result to communication device 4. The control unit further determines exercise advice suitable for the subject, based on the evaluation result and transmits the determined exercise advice to communication device 4. Communication device 4 displays the evaluation result of movement ability and the exercise advice transmitted from server 8 on the display.

The embodiments disclosed here should be understood as being illustrative rather than being limitative in all respects. The scope of the present invention is shown not in the foregoing description but in the claims, and it is intended that all modifications that come within the meaning and range of equivalence to the claims are embraced here.

### REFERENCE SIGNS LIST

1 acceleration sensor, 2 movement ability evaluating apparatus, 3 storage medium, 4 communication device, 6 network, 8 server, 10 sensor unit, 12, 42 CPU, 14, 22 memory, 16, 40 communication unit, 18, 44 circuit board, 20, 46 power supply, 24 signal processing circuit, 26, 60 radio signal receiver, 28, 62 radio signal transmitter, 30 file output unit, 48 display, 50 operation accepting unit, 64 control device, 68 storage device, 70 evaluation unit, 72 determination unit, 100 movement ability evaluating system, M subject.

1-16. (canceled)

17. A movement ability evaluating apparatus configured to evaluate a movement ability of a subject, the movement ability evaluating apparatus comprising:
a communication unit configured to acquire a front-back acceleration, a right-left acceleration, and an up-down acceleration measured by an acceleration sensor attached to a waist of the subject, during movement of the subject; and
a control unit configured to evaluate the movement ability of the subject, based on a temporal change of the front-back acceleration, the right-left acceleration, and the up-down acceleration acquired by the communication unit, wherein
the movement ability includes at least one of a front-back balance, a weight shift, and a right-left balance during movement of the subject, and
the control unit is configured to execute at least one of the following corresponding to the movement ability:
(a) calculating an indicator indicating the front-back balance, based on a frequency distribution of forward acceleration and backward acceleration in a temporal waveform of the front-back acceleration in at least one walking cycle;
(b) searching for a heel-contact time and a mid-stance time of one foot of the subject in a temporal waveform of the front-back acceleration in one walking cycle, and calculating an indicator indicating the weight shift, using a temporal waveform of the up-down acceleration in a period of time from the heel-contact time to the mid-stance time; and
(c) searching for a right-heel contact time, a right mid-stance time, a left-heel contact time, and a left mid-stance time of the subject in a temporal waveform of the front-back acceleration, based on a temporal wave form of the front-back acceleration and the right-left acceleration in one walking cycle, and calculating an indicator indicating the right-left balance, using a temporal waveform of leftward acceleration in a period of time from the right-heel contact time to the right mid-stance time and a temporal waveform of rightward acceleration from the left-heel contact time to the left mid-stance time.

18. A movement ability evaluating apparatus configured to evaluate a movement ability of a subject, the apparatus comprising:
a communication unit configured to acquire a front-back acceleration, a right-left acceleration, and an up-down acceleration measured by an acceleration sensor attached to a waist of the subject, during movement of the subject; and
a control unit configured to evaluate the movement ability of the subject, based on temporal change of the front-back acceleration, the right-left acceleration, and the up-down acceleration acquired by the communication unit, wherein
the movement ability includes at least one of a front-back balance, a weight shift, and a right-left balance during movement of the subject, and
the control unit is configured to execute at least one of the following corresponding to the movement ability:
(a) calculating an indicator indicating the front-back balance, using a delay time waveform of an autocorrelation function of the front-back acceleration;
(b) calculating an indicator indicating the weight shift, using a delay time waveform of an autocorrelation function of the up-down acceleration; and
(c) calculating an indicator indicating the right-left balance, using a delay time waveform of an autocorrelation function of the right-left acceleration and a delay time waveform of an autocorrelation function of the front-back acceleration.

19. The movement ability evaluating apparatus according to claim 17, wherein
the control unit is configured to calculate at least the indicator indicating the front-back balance, and
the control unit is configured to calculate an indicator indicating the front-back balance, based on a ratio between a total forward value determined by summing frequencies of the forward acceleration and a total backward value determined by summing frequencies of the backward acceleration in a histogram of the front-back acceleration in at least one walking cycle.

20. The movement ability evaluating apparatus according to claim 17, wherein the control unit is configured to calculate at least an indicator indicating the weight shift, and
the control unit is configured to
search a stepping-motion time immediately after heel contact and a stepping-motion time immediately after ball-of-foot contact in a temporal waveform of the up-down acceleration in a period of time from the heel-contact time to the mid-stance time, and
calculate an indicator indicating the weight shift of the one foot, based on a temporal waveform of the up-down acceleration around the stepping-motion time immediately after the heel contact and the stepping-motion time immediately after the ball-of-foot contact.

21. The movement ability evaluating apparatus according to claim 17, wherein
the control unit is configured to calculate at least an indicator indicating the right-left balance, and
the control unit is configured to calculate an indicator indicating the right-left balance, based on a ratio between an integral value by time-integrating the leftward acceleration in a period of time from a right-heel contact time to a right mid-stance time, and an integral value by time-integrating the rightward acceleration in a period time from a left-heel contact time to a left mid-stance time.

22. The movement ability evaluating apparatus according to claim 18, wherein
the control unit is configured to calculate at least an indicator indicating the front-back balance, and
the control unit is configured to calculate an indicator indicating the front-back balance, based on a deviation of a valley portion positioned between an origin and a first-peak position in the autocorrelation function of the front-back acceleration from an approximate curve obtained by approximating the valley portion to a quadric curve.

23. The movement ability evaluating apparatus according to claim 18, wherein
the control unit is configured to calculate at least an indicator indicating the weight shift, and
the control unit is configured to calculate an indicator indicating the weight shift, based on a ratio between a value at an origin of the autocorrelation function of the up-down acceleration and a value at a first peak position of the autocorrelation function of the up-down acceleration.

24. The movement ability evaluating apparatus according to claim 18, wherein the control unit is configured to calculate at least an indicator indicating the right-left balance, and
the control unit is configured to
search a first peak position and a second peak position of the autocorrelation function of the front-back acceleration,
search a first value at a peak position corresponding to the first peak position and a second value at a peak position corresponding to the second peak position in the autocorrelation function of the right-left acceleration, and
calculate an indicator indicating the right-left balance, based on a ratio between the first value and the second value.

25. The movement ability evaluating apparatus according to claim 17, wherein the control unit is configured to determine exercise advice suitable for the subject, based on an indicator indicating the movement ability.

26. The movement ability evaluating apparatus according to claim 25, further comprising a display configured to display at least one of an evaluation result of the control unit and the exercise advice.

27. A movement ability evaluating system comprising:
an acceleration sensor attached to a waist of a subject; and
a movement ability evaluating apparatus configured to evaluate a movement ability of the subject, based on a signal output by the acceleration sensor,
the movement ability evaluating apparatus including
a communication unit configured to acquire a front-back acceleration, a right-left acceleration, and an up-down acceleration measured by the acceleration sensor, during movement of the subject, and
a control unit configured to evaluate the movement ability, based on a temporal change of the front-back acceleration, the right-left acceleration, and the up-down acceleration acquired by the communication unit,
the movement ability including at least one of a front-back balance, a weight shift, and a right-left balance during movement of the subject, and
the control unit being configured to execute at least one of the following corresponding to the movement ability:
(a) calculating an indicator indicating the front-back balance, based on a frequency distribution of forward acceleration and backward acceleration in a temporal waveform of the front-back acceleration in at least one walking cycle;
(b) searching for a heel-contact time and a mid-stance time of one foot of the subject, in a temporal waveform of the front-back acceleration in one walking cycle, and calculating an indicator indicating the weight shift, using a temporal waveform of the up-down acceleration in a period of time from the heel-contact time to the mid-stance time; and
(c) searching for a right-heel contact time, a right mid-stance time, a left-heel contact time, and a left mid-stance time of the subject in a temporal waveform of the front-back acceleration, based on a temporal wave form of the front-back acceleration and the right-left acceleration in one walking cycle, and calculating an indicator indicating the right-left balance, using a temporal waveform of leftward acceleration in a period of time from the right-heel contact time to the right mid-stance time and a temporal waveform of rightward acceleration from the left-heel contact time to the left mid-stance time.

28. The movement ability evaluating system according to claim 27, wherein the acceleration sensor includes
a sensor unit configured to measure the front-back acceleration, the right-left acceleration, and the up-down acceleration produced at the waist of the subject, and
a signal processing circuit configured to correct a measured value of the sensor unit when the subject is standing still to a zero point of the front-back acceleration, the right-left acceleration, and the up-down acceleration and to acquire a measured value of the sensor unit at intervals of 1 ms to 200 ms during movement of the subject.

29. The movement ability evaluating system according to claim 28, wherein
the movement ability evaluating apparatus further includes a storage device configured to store the front-back acceleration, the right-left acceleration, and the up-down acceleration acquired by the communication unit and an evaluation result in the control unit, and
the acceleration sensor includes
a transmitter configured to transmit a measured value of the sensor unit acquired by the signal processing circuit to the communication unit, and
a memory configured to save the measured value of the sensor unit acquired by the signal processing circuit, and
the signal processing circuit configured to select one of the storage device and the memory in accordance with a signal from the movement ability evaluating apparatus to save the measured value of the sensor unit.

30. A movement ability evaluating system comprising:
an acceleration sensor attached to waist of a subject; and
a movement ability evaluating apparatus configured to evaluate a movement ability of the subject, based on a signal output by the acceleration sensor,
the movement ability evaluating apparatus including
a communication unit configured to acquire a front-back acceleration, a right-left acceleration, and an up-down acceleration measured by an acceleration sensor attached to waist of the subject, during movement of the subject, and
a control unit configured to evaluate the movement ability, based on temporal change of the front-back acceleration, the right-left acceleration, and the up-down acceleration acquired by the communication unit,
the movement ability including at least one of a front-back balance, a weight shift, and a right-left balance during movement of the subject, and
the control unit configured to execute the following corresponding to the movement ability:
(a) calculating an indicator indicating the front-back balance, using a delay time waveform of an autocorrelation function of the front-back acceleration;
(b) calculating an indicator indicating the weight shift, using a delay time waveform of an autocorrelation function of the up-down acceleration; and
(c) calculating an indicator indicating the right-left balance, using a delay time waveform of an autocorrelation function of the right-left acceleration and a delay time waveform of an autocorrelation function of the front-back acceleration.
